(19) United States
(12) Patent Application Publication
(10) Pub. No.: US 2022/0008215 A1
(43) Pub. Date: Jan. 13, 2022

(54) SPINAL CAGE WITH KEEL

(71) Applicant: CTL Medical Corporation, Addison, TX (US)

(72) Inventors: Sean SUH, Milltown, NJ (US); Jon SUH, Ambler, PA (US); Alex WENDEL, Dallas, TX (US); Jeffrey WANG, Los Angeles, CA (US)

(21) Appl. No.: 17/370,874

(22) Filed: Jul. 8, 2021

Related U.S. Application Data
(63) Continuation-in-part of application No. 17/369,856, filed on Jul. 7, 2021.
(60) Provisional application No. 63/049,286, filed on Jul. 8, 2020.
(60) Provisional application No. 63/049,286, filed on Jul. 8, 2020.

(51) Int. Cl. A61F 2/44; A61F 2/30

(52) U.S. Cl. CPC A61F 2/4455; A61F 2/30771; A61F 2002/30904; A61F 2002/30322; A61F 2002/30827; A61F 2002/30604; A61F 2002/30884

(57) ABSTRACT

Disclosed are modular anchoring adaptors that can engage with existing features of various spinal cages to help eliminate migration and/or anterior expulsion cause by the instability of the spine, such as lordosis. Such modular adaptors will desirably mate or engage with existing features to enhance anchoring within the vertebral disc space.

### CROSS-REFERENCE TO RELATED APPLICATIONS

This application is a continuation-in-part of U.S. patent application Ser. No. 17/369,856 entitled “Cage With Keel” filed Jul. 7, 2021, which in turn claims the benefit of U.S. Provisional Application No. 63/049,286 entitled “Cage with Keel” filed Jul. 8, 2020, the disclosures of which are each incorporated by reference herein in their entireties.

### TECHNICAL FIELD

The invention relates to methods, devices, and systems for an improved spinal cage with modular anchoring elements to help eliminate migration and/or anterior expulsion caused by scoliosis, kyphosis, lordosis, spinal stenosis, etc. More specifically, the invention relates to a modular anchoring adaptor that engages with existing features of various interspinal implants, including commercially available ALIF cages, to help eliminate migration and/or anterior expulsion, especially where space may be limited.

### BACKGROUND OF THE INVENTION

The spinal column of vertebrates provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain and can sustain permanent neurological damage if the conditions are not treated appropriately.

Alternatively, or in addition, there are several types of spinal curvature disorders. Examples of such spinal curvature disorders include, but need not be limited to, lordosis, kyphosis and scoliosis.

One technique of treating spinal disorders, in particular the degenerative, traumatic and/or congenital issues, is via surgical arthrodesis of the spine. This can be accomplished by removing the intervertebral disk and replacing it with implant(s) and/or bone and immobilizing the spine to allow the eventual fusion or growth of the bone across the disk space to connect the adjoining vertebral bodies together. The stabilization of the vertebra to allow fusion is often assisted by the surgically implanted device(s) to hold the vertebral bodies in proper alignment and allow the bone to heal, much like placing a cast on a fractured bone. Such techniques have been effectively used to treat the above-described conditions and in most cases are effective at reducing the patient's pain and preventing neurological loss of function.

The spinal curvature disorders and/or contour issues present on the surfaces of the vertebrae may present additional challenges. As such, there is need for further improvement, and the present subject matter is such improvement. Currently, traditional ALIF cages contain ineffective designs to help reduce or eliminate migration and/or anterior expulsion. Typically, surgeons insert a screw on the anterior face of the vertebrae directed diagonally to allow the screw head to partially occlude the anterior face of the ALIF cage. Alternatively, there are other ALIF designs that require a full plate that is coupled to the anterior face of the upper and lower vertebrate with additional insertion tools and fixation screws, which the plate does not engage with the implanted ALIF design. These traditional designs are less cost effective and may not be useful when there is only a limited amount of space available.

### BRIEF SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

Disclosed is an implant device for the spine, the implant device being desirably sized and configured for location between two adjacent vertebrae. Various features of the present invention include the realization of a need for a variety of anchoring systems and/or components that can be fixed and/or modularly connected to an interbody spinal cage or other implant to desirably reduce and/or minimize movement of the cage or other implant relative to the adjacent anatomical surfaces. In many embodiments, the modular anchoring component can incorporate a various of designs and/or configurations that can accommodate various anatomical constraints and/or conditions, including unique anatomical conditions that may facilitate and/or constrain one or more attachment modalities and/or portions thereof. In some embodiments, a surgical kit containing a plurality of fixed and/or modular anchoring components that can each be attached to an interbody spinal cage or other implant (with a plurality of such interbody spinal cages or other implants optionally included in the kit) can greatly reduce the number and/or extent of kit components necessary for a given surgery, with commensurate reductions in the cost and/or complexity of the implant kit.

In one exemplary embodiment, a double keel ALIF cage system comprises: a cage, a first keel and a second keel. The cage including a longitudinal axis, a superior surface, an inferior surface, an anterior surface, a posterior surface, medial surface and/or a lateral surface. The first keel is disposed into the superior surface, extending between the anterior and posterior surfaces. The second keel is disposed onto the inferior surface, extending between the anterior and posterior surfaces. The first keel may be aligned and/or offset from the second keel. The first and/or second keel may comprise one or more teeth, the one or more teeth may be solid. Alternatively, the one or more teeth and/or each of the one or more teeth may comprise capillary through holes. The anterior surface of the cage comprises a first opening and a second opening. The first opening and/or second opening may comprise threads, the opening sized and configured for a fixation screw. The first opening and/or second opening may be sized and configured for a dowel pin. The anterior surface of the first and/or second keels may be flush with the anterior surface of the cage. Alternatively, the anterior surface of the first and/or second keels may be offset with the anterior surface of the cage. Furthermore, the medial and/or lateral surfaces of the cage may further comprise a channel. Also, the cage may further comprise a cage opening extending from the superior surface through the inferior surface. In one exemplary embodiment, an ALIF cage system can be sized and configured to fit between the adjacent vertebral bodies.

In one exemplary embodiment, a triple keel ALIF cage system comprises: a cage, a first keel, a second keel and a third keel. The cage including a longitudinal axis, a superior surface, an inferior surface, an anterior surface, a posterior surface, medial surface and/or a lateral surface. The first keel and the second keel are spaced apart disposed into the superior surface, extending between the anterior and posterior surfaces. The third keel is disposed onto the inferior surface, extending between the anterior and posterior surfaces, and positioned between the first and second keels. The first, second and/or third keel may comprise one or more teeth, the one or more teeth may be solid. Alternatively, the one or more teeth and/or each of the one or more teeth may comprise capillary through holes. The anterior surface of the cage comprises a first opening and a second opening. The first opening and/or second opening may comprise threads, the opening sized and configured for a fixation screw. The first opening and/or second opening may be sized and configured for a dowel pin. The anterior surface of the first, second and/or third keels may be flush with the anterior surface of the cage. Alternatively, the anterior surface of the first, and/or third second keels may be offset with the anterior surface of the cage. Furthermore, the medial and/or lateral surfaces of the cage may further comprise a side channel. Also, the cage may further comprise a cage opening extending from the superior surface through the inferior surface. The ALIF cage system is sized and configured to fit between the adjacent vertebral bodies.

In one exemplary embodiment, a quad keel ALIF cage system comprises: a cage, a first keel set and a second keel set. The cage including a longitudinal axis, a superior surface, an inferior surface, an anterior surface, a posterior surface, medial surface and/or a lateral surface. The first keel set is disposed into the superior surface, extending between the anterior and posterior surfaces. The second keel set is disposed onto the inferior surface, extending between the anterior and posterior surfaces. The first keel set may be aligned and/or offset from the second keel. The first and/or second keel set may comprise one or more teeth, the one or more teeth and/or each of the one or more teeth may be solid. Alternatively, the one or more teeth and/or each of the one or more teeth may comprise capillary through holes. The anterior surface of the cage comprises a first opening and a second opening. The first opening and/or second opening may comprise threads, the opening sized and configured for a fixation screw. The first opening and/or second opening may be sized and configured for a dowel pin. The anterior surface of the first and/or second keels set may be flush with the anterior surface of the cage. Alternatively, the anterior surface of the first and/or second keel set may be offset with the anterior surface of the cage. Furthermore, the medial and/or lateral surfaces of the cage may further comprise a side channel. Also, the cage may further comprise a cage opening extending from the superior surface through the inferior surface. The ALIF cage system is sized and configured to fit between the adjacent vertebral bodies.

In one exemplary embodiment, a modular ALIF cage system comprises: a cage, and an insert. The cage including a longitudinal axis, a superior surface, an inferior surface, an anterior surface, a posterior surface, medial surface and/or a lateral surface. The cage further comprising a cage opening extending through the superior to inferior surfaces. The cage may further comprise a side opening, the side opening extending from the medial to lateral surfaces of the cage. The anterior surface of the cage comprises a first opening and a second opening. The first opening and/or second opening may comprise threads, the opening sized and configured for a fixation screw. The first opening and/or second opening may be sized and configured for a dowel pin. The modular insert is removable connected within the cage opening, the cage opening is sized and configured to receive the insert. The insert matches or substantially matches the contours of the cage opening. The modular insert comprises one or more keels. The one or more keels may be disposed onto the superior surface and/or inferior surface, extending between the anterior and posterior surfaces. The one or more keels may be spaced apart from each other. The one or more keels may be aligned and/or offset from the adjacent keel. The one or more keels may comprise one or more teeth, the one or more teeth may be solid and/or each of the one or more teeth may be solid. Alternatively, the one or more teeth and/or each of the one or more teeth may comprise capillary through holes. The anterior surface of the cage comprises a first opening and a second opening. The first opening and/or second opening may comprise threads, the opening sized and configured for a fixation screw. The first opening and/or second opening may be sized and configured for a dowel pin. The anterior surface of the first and/or second keels may be flush with the anterior surface of the cage. Alternatively, the anterior surface of the first and/or second keels may be offset with the anterior surface of the cage. Furthermore, the medial and/or lateral surfaces of the cage may further comprise a side channel. The ALIF cage system is sized and configured to fit between the adjacent vertebral bodies.

In one exemplary embodiment, a modular ALIF cage system comprises: a cage, one or more keels, and a frontal plate. The cage including a longitudinal axis, a superior surface, an inferior surface, an anterior surface, a posterior surface, medial surface and/or a lateral surface. The cage further comprising a cage opening extending through the superior to inferior surfaces. The superior and/or inferior surfaces of the cage further comprising a one or more keel channels, the one or more keel channels comprises a pin opening, the pin opening is sized and configured to receive dowel pins. The cage may further comprise a side opening, the side opening extending from the medial to lateral surfaces of the cage. The anterior surface of the cage comprises plate channel, a first opening and a second opening. The first opening and/or second opening may comprise threads, the opening sized and configured for a fixation screw. The first opening and/or second opening may be sized and configured for a dowel pin. The one or more keels are removably connected within the one or more keel channels, the one or more keel channels are sized and configured to receive the one or more keels. The one or more keel channels matches or substantially matches the contours of the one or more keels. The one or more keels extending between the anterior and posterior surfaces. The one or more keels may be spaced apart from each other. The one or more keels may be aligned and/or offset from the adjacent keel. The one or more keels may comprise one or more teeth, the one or more teeth may be solid and/or each of the one or more teeth may be solid. Alternatively, the one or more teeth and/or each of the one or more teeth may comprise capillary through holes. The anterior surface of the cage comprises a first opening and a second opening. The first opening and/or second opening may comprise threads, the opening sized and configured for a fixation screw. The first opening and/or second opening may be sized and configured for a dowel pin. The anterior surface of the one or more keels may be flush with the anterior surface of the cage. Alternatively, the anterior surface of the one or more keels may be offset with the anterior surface of the cage. Furthermore, the medial and/or lateral surfaces of the cage may further comprise a side channel extending between the superior and inferior surfaces. The frontal plate disposed into the plate channel of the cage and is removably connected. The frontal plate further comprises a plurality of openings that are spaced apart. The plurality of openings of the frontal plate are concentrically aligned with the first and/or second openings of the cage. The ALIF cage system is sized and configured to fit between the adjacent vertebral bodies.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings.

FIG. 1 depicts a front view of one embodiment of a double keel cage system inserted between vertebral bodies;

FIGS. 2A-2B depicts an isometric and top view of an alternate embodiment of a double keel cage;

FIG. 3A-3B depicts an isometric and top view of an alternate embodiment of a double keel cage;

FIGS. 4A-4B depicts an isometric and top view of an alternate embodiment of a double keel cage;

FIG. 5 depicts an isometric view of an alternate of a double keel cage;

FIG. 6 depicts an front view of one embodiment of a triple keel cage system inserted between vertebral bodies;

FIGS. 7A-7C depicts a multiple views of one embodiment of a triple keel cage;

FIG. 7D depicts a cross-section view of the triple keel cage of FIGS. 7A-7C;

FIG. 8A depicts a cross-section view of the triple keel cage of FIGS. 8B-8C;

FIGS. 8B-8C depicts a top and bottom view of an alternate embodiment of a modular adapter;

FIG. 9 depicts an front view one embodiment of a quad keel cage system inserted between vertebral bodies;

FIGS. 10A-10B depicts an isometric and top view of an alternate embodiment of a quad keel cage;

FIG. 11 depicts a top view of an alternate embodiment of a quad keel cage;

FIG. 12A depicts an exploded view of one embodiment of a modular keel cage assembly;

FIGS. 12B-12C depicts a top and front view of one embodiment of the modular keel cage assembly of FIG. 12A;

FIGS. 13A-13B depicts isometric views of an alternate embodiment of the modular keel cage assembly;

FIG. 13C depicts an exploded view of the modular keel cage assembly of FIGS. 13A-13B;

FIG. 13D depicts a perspective view of another alternative embodiment of a modular keel element;

FIG. 14A depicts another alternative embodiment of a modular keel cage with offset keels;

FIG. 14B depicts the modular keel cage of FIG. 14A in a single level construct;

FIG. 14C depicts the modular keel cage of FIG. 14A in a multi-level construct;

FIG. 14D depicts another embodiment of a modular keel cage that includes angled end plates that provide for lordotic adjustment of the treated spinal level;

FIG. 14E depicts a side view of the modular keel cage of FIG. 14D implanted in a spinal level;

FIG. 14F depicts a side view of another alternative embodiment of a modular keel cage without angled endplates implanted in a spinal level;

FIG. 15 depicts another alternative embodiment of a modular keel cage with offset keel elements;

FIG. 16 depicts another alternative embodiment of a modular keel cage with offset keel elements;

FIGS. 17A and 17B depict perspective and front plan views of another alternative embodiment of a modular keel cage with offset keel elements and an anterior impaction plate;

FIG. 18 depicts another alternative embodiment of a modular keel cage with offset keel elements

FIGS. 19A and 19B depict side and top plans views of another alternative embodiment of a modular keel cage with elongated keel elements;

FIG. 20 depicts an exemplary cage assembly with modular elements;

FIG. 21 depicts another exemplary cage assembly with modular elements;

FIG. 22 depicts various embodiments of vertebral body replacements incorporating modular elements;

FIG. 23 depicts front plan views of various keel positions that can be accommodated by a single modular keel system;

FIG. 24 depicts various embodiments of interbody spacers incorporating modular keel elements; and

FIG. 25 depicts various surgical approaches that can be utilized with the various modular cage embodiments described herein.

### DETAILED DESCRIPTION OF THE INVENTION

The present subject matter relates generally to devices for the fixation and support of vertebrae. In particular, the present subject matter relates to implant devices that can incorporate a variety of modular anchoring components. In various embodiments, the anchoring components are modularly connected to an interbody spinal cage or other implant to desirably reduce and/or minimize movement of the cage or other implant relative to the adjacent anatomical surfaces. In many embodiments, the modular anchoring component can incorporate a various of designs and/or configurations that can accommodate various anatomical constraints and/or conditions, including unique anatomical conditions that may facilitate and/or constrain one or more attachment modalities and/or portions thereof. In some embodiments, a surgical kit containing a plurality of modular anchoring components that can each be attached to an interbody spinal cage or other implant (with a plurality of such interbody spinal cages or other implants optionally included in the kit) can greatly reduce the number and/or extent of kit components necessary for a given surgery, with commensurate reductions in the cost and/or complexity of the implant kit.

As is known in the art, a spinal column desirably provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain, and can sustain permanent neurological damage if the conditions are not treated appropriately.

Alternatively or in addition, there are several types of spinal curvature disorders. Examples of such spinal curvature disorders include, but need not be limited to, lordosis, kyphosis and scoliosis.

Aside from the challenges presented by the limited access, size and angulation of various surgical approaches to the spinal anatomy, spinal curvature disorders and/or contour issues can often present on the surfaces of the vertebrae, which may present additional challenges.

The present subject matter will now be described with reference to the drawings, wherein like reference numerals are used to refer to like elements throughout. It is to be appreciated that the various drawings are not necessarily drawn to scale from one figure to another nor inside a given figure, and in particular that the size of the components are arbitrarily drawn for facilitating the understanding of the drawings. In the following description, for purposes of explanation, numerous specific details are set forth in order to provide a thorough understanding of the present subject matter. It may be evident, however, that the present subject matter can be practiced without these specific details. Additionally, other embodiments of the subject matter are possible and the subject matter is capable of being practiced and carried out in ways other than as described. The terminology and phraseology used in describing the subject matter is employed for the purpose of promoting an understanding of the subject matter and should not be taken as limiting.

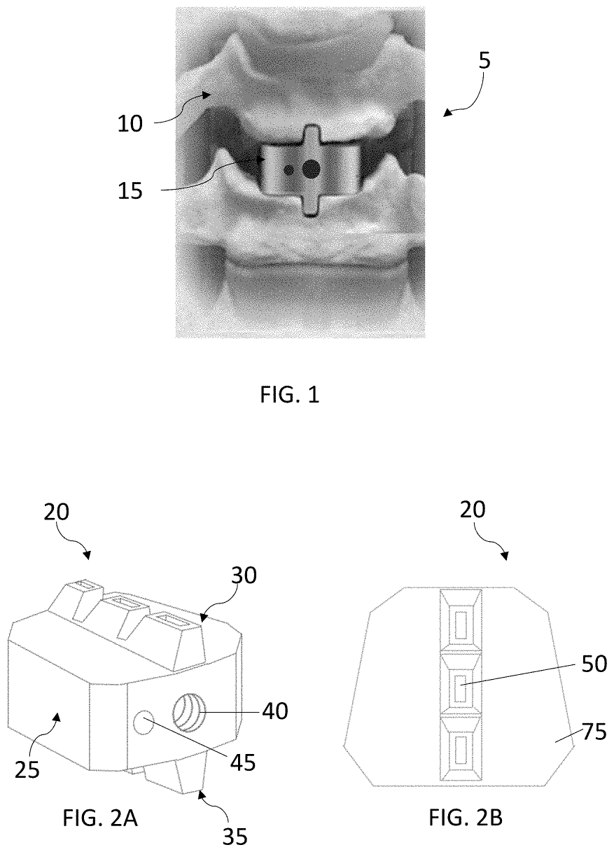

FIG. 1 depict one embodiment of an improved ALIF cage system 10 that is secured between vertebral bodies 5. The ALIF cage system 10 comprises keels, which is adapted to stabilize the spine. Keels are commonly used within other types of spinal implants, such as motion preservation implants, but are not considered for fusion implants. Keels beneficially provide an improved mechanical interlock with the adjacent vertebral bodies, achieves improved fixation of the spinal implant to patient bone, and promotes additional bony fusion between the adjacent vertebrae.

FIGS. 2A-2B, 3A-3B, 4A-4B and 5 comprises various embodiments of dual or double keeled ALIF cage system 20, 55, 70, 95. In one exemplary embodiment, a double keel ALIF cage system 20, 55, 70, 95 comprises: a cage 25, a first keel 30 and a second keel 35. The cage 25 including a longitudinal axis 15, a superior surface 75, an inferior surface 80, an anterior surface 63, a posterior surface 68, medial surface 73 and/or a lateral surface 78. The first keel 30 is disposed into the superior surface 75, extending between the anterior 63 and posterior surfaces 68. The second keel 35 is disposed onto the inferior surface 80, extending between the anterior 63 and posterior surfaces 68. The first keel 30 may be aligned and/or offset from the second keel 35. The first keel 30 and/or the second keel may be aligned with the longitudinal axis 15 and/or offset from the longitudinal axis 15. The first 30 and/or second keel 35 may comprise one or more teeth 33, the one or more teeth 33 may be solid. Alternatively, the one or more teeth 33 and/or each of the one or more teeth 33 may comprise capillary through holes 50. Each of the one or more teeth 33 of the first keel 30 and the second keel 35 comprising a rectangular or trapezoidal shape, and the first keel 30 and the second keel extending upwardly from the superior 63 and/or inferior surfaces 68.

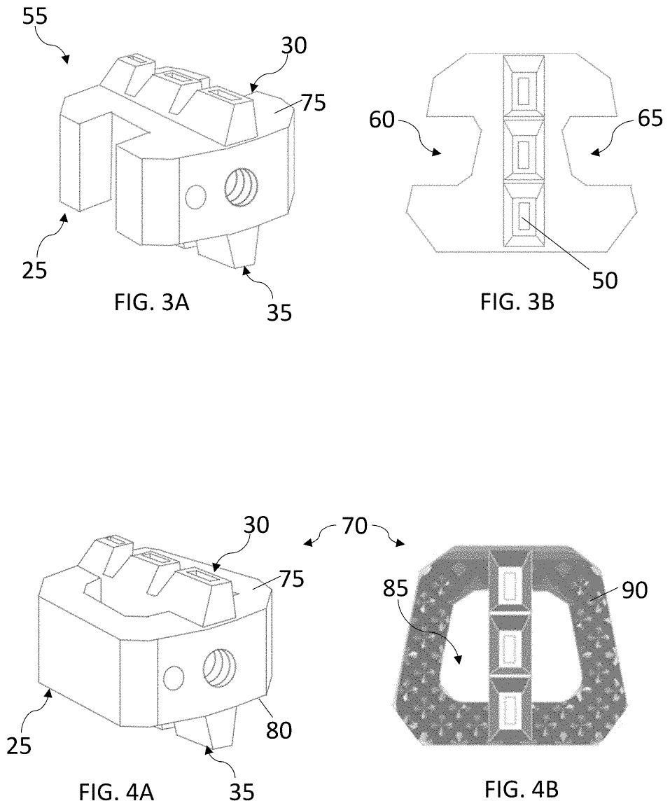

The anterior surface 63 of the cage 25 comprises a first opening 40 and a second opening 45. The first opening 40 and/or second opening 45 may comprise threads, the opening sized and configured for a fixation screw. The first opening 40 and/or second opening 45 may be sized and configured for a dowel pin (not shown). The anterior surface 63 of the first 30 and/or second keels 35 may be flush with the anterior surface 63 of the cage 25. Alternatively, the anterior surface 63 of the first 30 and/or second keels 35 may be offset with the anterior surface 63 of the cage 25. Furthermore, the medial 73 and/or lateral surfaces 78 of the cage may further comprise a side channel 60, 65 as shown in FIG. 3A-3B. The side channel 60,65 may extend between the superior surface 75 and the inferior surface 80. The side channels 60, 65 may engage with tools or other inserts.

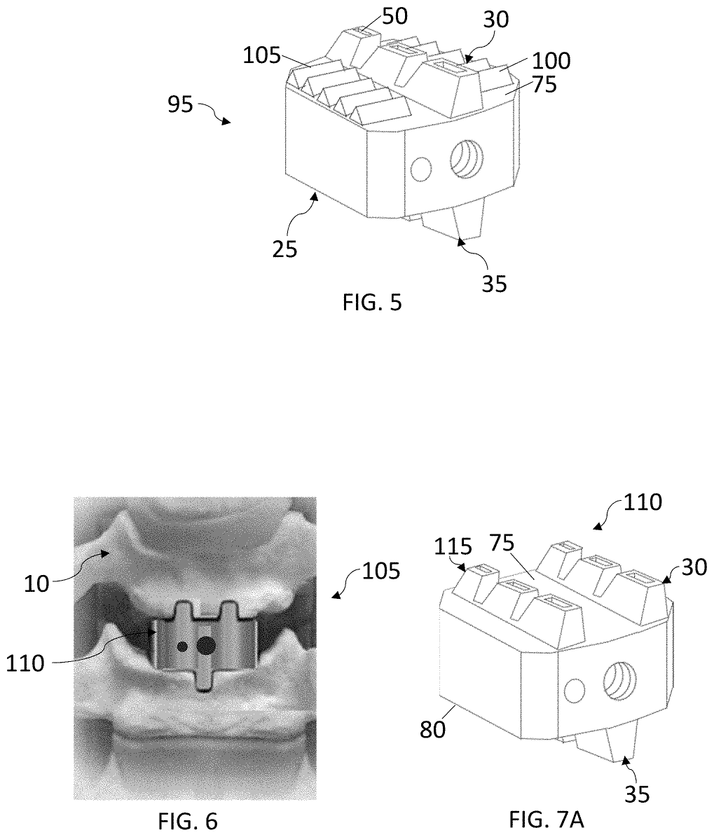

The cage 25 may further comprise a cage opening 85 extending from the superior surface 75 through the inferior surface 80 as shown in FIG. 4A-4B. At least a portion of the first keel 30 and the second keel 35 may be disposed over the cage opening 85. At least one of the superior surface 75, an inferior surface 80, an anterior surface 63, a posterior surface 68, medial surface 73 and/or a lateral surface 78 of the cage 25 may further comprise surface texturing 90, and/or any combination thereof as shown in FIGS. 4B, 8B-8C, 11, and 12A-12C. The surface texturing 90 may comprise a surface ingrowth polymer and/or ceramics and/or a surface ingrowth metal, as well as a macro or micro-texturing of the surfaces of the implant to desirably increase surface area for better adhesion, as well as promote bone ingrowth into the surface of device. Accordingly, the cage 25 may further comprise a plurality of serrations 100, 105 as shown in FIG. 5. The plurality of serrations 100, 105 may be disposed adjacent to the first keel 30 and the second keel 35. The plurality of serrations 100, 105 may be disposed on the medial and/or lateral sides of the first keel 30 and the second keel 35. The plurality of serrations 100, 105 may be disposed on the superior 75 and/or inferior surfaces 80 extending upwardly.

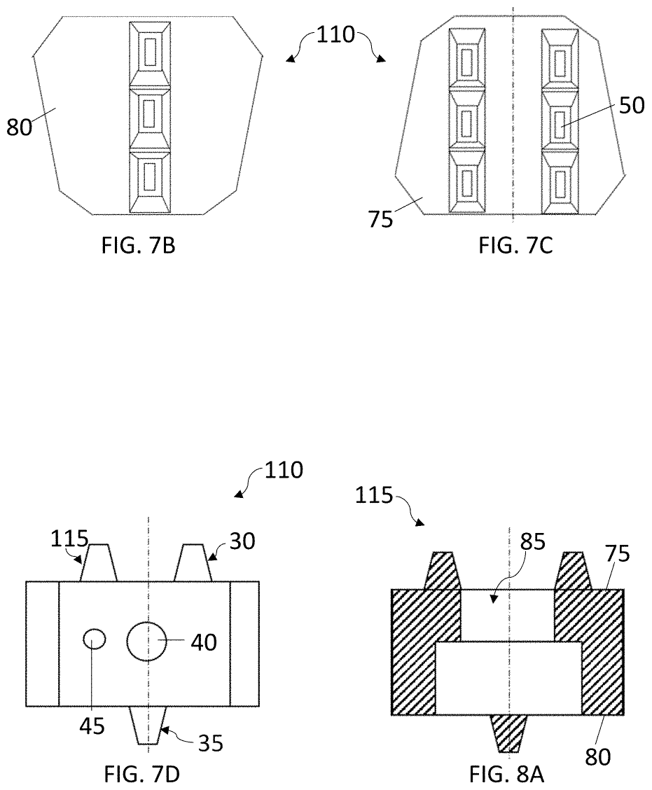

FIGS. 6, 7A-7D, and 8A-8C comprises various embodiments of a triple keeled ALIF cage system 110, 120. In one exemplary embodiment, a triple keel ALIF cage system 110, 120 comprises: a cage 25, a first keel 30, a second keel 35 and a third keel 115. The cage 25 including a longitudinal axis 15, a superior surface 75, an inferior surface 80, an anterior surface 63, a posterior surface 68, medial surface 73 and/or a lateral surface 78. The first keel 30 and the third keel 115 are spaced apart and disposed into the superior surface 75, extending between the anterior 63 and posterior surfaces 68. The second keel 35 is disposed onto the inferior surface 80, extending between the anterior 63 and posterior surfaces 68. The second keel 35 may be disposed between the first keel 30 and the third keel 115. The second keel 35 may be aligned with the longitudinal axis 15 and/or offset from the longitudinal axis 15. The first 30, second keel 35 and the third keel 115 may comprise one or more teeth 33, the one or more teeth 33 may be solid. Alternatively, the one or more teeth 33 and/or each of the one or more teeth 33 may comprise capillary through holes 50. Each of the one or more teeth 33 of the first keel 30, the second keel 35 and/or the third keel 115 comprising a rectangular or trapezoidal shape, and the first keel 30, the second keel 35 and/or the third keel 115 extending upwardly from the superior 63 and/or inferior surfaces 68.

The anterior surface 63 of the cage 25 comprises a first opening 40 and a second opening 45. The first opening 40 and/or second opening 45 may comprise threads, the opening sized and configured for a fixation screw. The first opening 40 and/or second opening 45 may be sized and configured for a dowel pin (not shown). The anterior surface 63 of the first 30 and/or second keels 35 may be flush with the anterior surface 63 of the cage 25. Alternatively, the anterior surface 63 of the first 30, second keel 35 and/or third keel 115 may be offset with the anterior surface 63 of the cage 25. Furthermore, the medial 73 and/or lateral surfaces 78 of the cage may further comprise a side channel 60, 65 as shown in FIG. 3A-3B. The side channel 60,65 may extend between the superior surface 75 and the inferior surface 80. The side channels 60, 65 may engage with tools or other inserts. The side channels 60, 65 may engage with tools or other inserts.

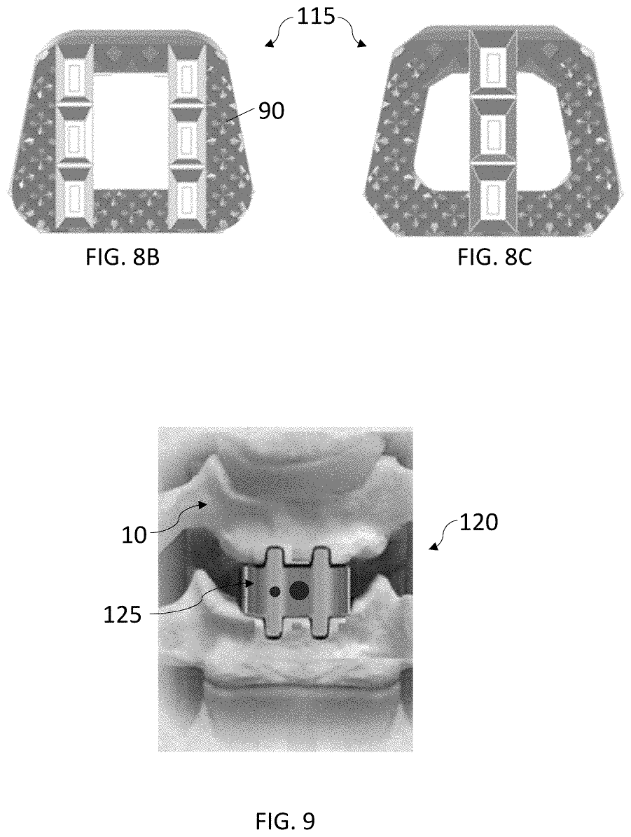

The cage 25 may further comprise a cage opening 85 extending from the superior surface 75 through the inferior surface 80 as shown in FIG. 4A-4B and FIG. 8A. The cage opening 85 may extend through the superior surface 75 to the inferior surface 80. The cage opening 85 may be uniform and/or non-uniform. The cage opening 85 may comprise a first cage opening 85a and a second cage opening 85b. The first cage opening 85a comprising a different width from the second cage opening 85b. At least a portion of the first keel 30, the second keel 35 and/or the third keel 115 may be disposed over the cage opening 85. At least one of the superior surface 75, an inferior surface 80, an anterior surface 63, a posterior surface 68, medial surface 73 and/or a lateral surface 78 of the cage 25 may further comprise surface texturing 90, and/or any combination thereof as shown in FIGS. 4B, 8B-8C, 11, and 12A-12C. Accordingly, the cage 25 may further comprise a plurality of serrations 100, 105 as shown in FIG. 5. The plurality of serrations 100, 105 may be disposed adjacent to the first keel 30 and the second keel 35. The plurality of serrations 100, 105 may be disposed on the medial and/or lateral sides of the first keel 30 and the second keel 35. The plurality of serrations 100, 105 may be disposed on the superior 75 and/or inferior surfaces 80 extending upwardly.

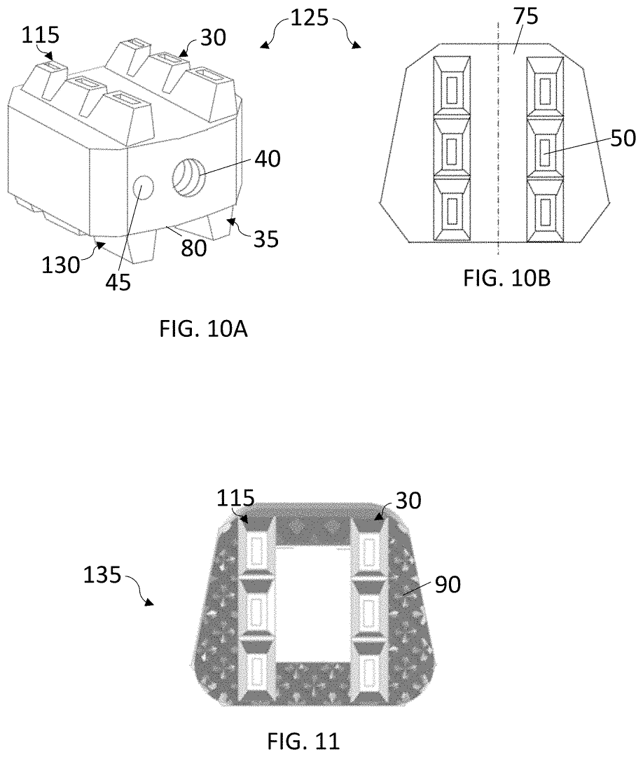

FIGS. 9, 10A-10B, and 11 comprises various embodiments of a quad keeled ALIF cage system 125, 135. In one exemplary embodiment, a quad keel ALIF cage system 125, 135 comprises: a cage 25, a first keel 30, a second keel 35, a third keel 115 and a fourth keel 130. The cage 25 including a longitudinal axis 15, a superior surface 75, an inferior surface 80, an anterior surface 63, a posterior surface 68, medial surface 73 and/or a lateral surface 78. The first keel 30 and the third keel 115 are spaced apart and disposed into the superior surface 75, extending between the anterior 63 and posterior surfaces 68. The second keel 35 and the fourth keel 130 is spaced apart and disposed onto the inferior surface 80, extending between the anterior 63 and posterior surfaces 68. The first keel 30 and the third keel 115 may be aligned or offset from the second keel 35 and fourth keel 130. The first 30, the second keel 35, the third keel 115 and/or the third keel 130 may comprise one or more teeth 33, the one or more teeth 33 may be solid. Alternatively, the one or more teeth 33 and/or each of the one or more teeth 33 may comprise capillary through holes 50. Each of the one or more teeth 33 of the first keel 30, the second keel 35 and/or the third keel 115 comprising a rectangular or trapezoidal shape, and the first keel 30, the second keel 35 and/or the third keel 115 extending upwardly from the superior 63 and/or inferior surfaces 68.

The anterior surface 63 of the cage 25 comprises a first opening 40 and a second opening 45. The first opening 40 and/or second opening 45 may comprise threads, the opening sized and configured for a fixation screw. The first opening 40 and/or second opening 45 may be sized and configured for a dowel pin (not shown). The anterior surface 63 of the first 30 and/or second keels 35 may be flush with the anterior surface 63 of the cage 25. Alternatively, the anterior surface 63 of the first 30, second keel 35, third keel 115 and/or fourth keel 130 may be offset with the anterior surface 63 of the cage 25. Furthermore, the medial 73 and/or lateral surfaces 78 of the cage may further comprise a side channel 60, 65 as shown in FIG. 3A-3B. The side channel 60,65 may extend between the superior surface 75 and the inferior surface 80. The side channels 60, 65 may engage with tools or other inserts.

The cage 25 may further comprise a cage opening 85 extending from the superior surface 75 through the inferior surface 80 as shown in FIG. 4A-4B and FIG. 8A. The cage opening 85 may extend through the superior surface 75 to the inferior surface 80. The cage opening 85 may be uniform and/or non-uniform. The cage opening 85 may comprise a first cage opening 85a and a second cage opening 85b. The first cage opening 85a comprising a different width from the second cage opening 85b. At least a portion of the first keel 30, the second keel 35, the third keel 115 and/or the fourth keel 130 may be disposed over the cage opening 85. At least one of the superior surface 75, an inferior surface 80, an anterior surface 63, a posterior surface 68, medial surface 73 and/or a lateral surface 78 of the cage 25 may further comprise surface texturing 90, and/or any combination thereof as shown in FIGS. 4B, 8B-8C, 11, and 12A-12C. Accordingly, the cage 25 may further comprise a plurality of serrations 100, 105 as shown in FIG. 5. The plurality of serrations 100, 105 may be disposed adjacent to the keels 30, 35, 115, 130. The plurality of serrations 100, 105 may be disposed between the keels 30, 35, 115, 130. The plurality of serrations 100, 105 may be disposed on the medial and/or lateral sides of the keels 30, 35, 115, 130. The plurality of serrations 100, 105 may be disposed on the superior 75 and/or inferior surfaces 80 extending upwardly.

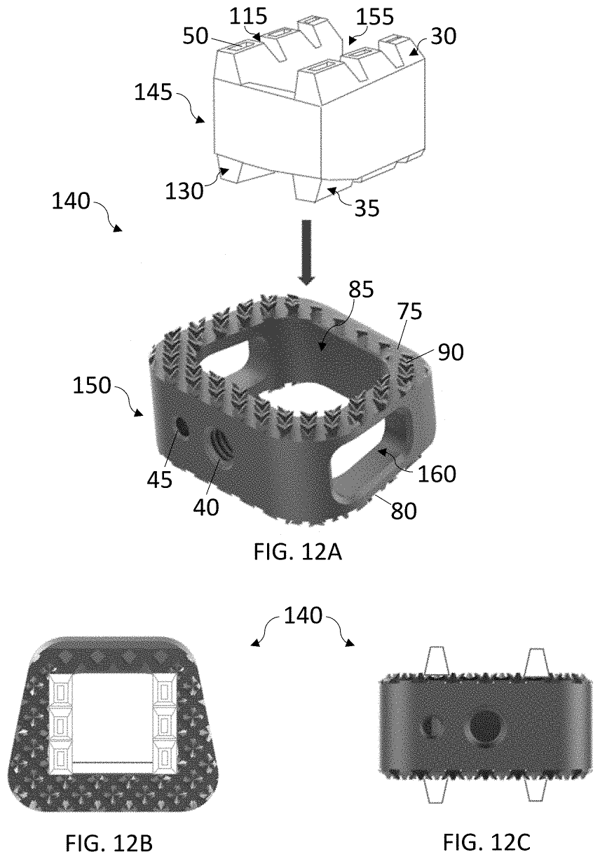

FIGS. 12A-12C depict a modular keel ALIF cage system 140. The modular keel ALIF cage system 140 comprises: a cage 25, and an insert 145. The cage 25 including a longitudinal axis 15, a superior surface 75, an inferior surface 80, an anterior surface 63, a posterior surface. The cage 25 may further comprise a cage opening 85 extending from the superior surface 75 through the inferior surface 80 as shown in FIG. 4A-4B, FIG. 8A and FIG. 12A-12C. The cage opening 85 may extend through the superior surface 75 to the inferior surface 80. The cage opening 85 may be uniform and/or non-uniform. The cage opening 85 may comprise a first cage opening 85a and a second cage opening 85b. The first cage opening 85a comprising a different width from the second cage opening 85b. Each of the cage openings 85, 85a, 85b comprising a shape, each of the cage openings shapes may be the same or they may be different.

The anterior surface 63 of the cage 25 comprises a first opening 40 and a second opening 45. The first opening 40 and/or second opening 45 may comprise threads, the opening sized and configured for a fixation screw. The first opening 40 and/or second opening 45 may be sized and configured for a dowel pin (not shown). The anterior surface 63 of the one or more keels 30, 35, 115, 130 of the modular insert 145 may be flush with the anterior surface 63 of the cage 25. Alternatively, the anterior surface 63 of the one or more keels 30, 35, 115, 130 of the modular insert 145 may be offset with the anterior surface 63 of the cage 25. Furthermore, the medial 73 and/or lateral surfaces 78 of the cage 25 may further comprise a side channel 60, 65 as shown in FIG. 3A-3B. The side channel 60,65 may extend between the superior surface 75 and the inferior surface 80. Alternatively, the medial 73 and/or lateral surface 78 of the cage 25 may further comprise one or more side openings 150,155. The side openings 150, 155 may engage with tools or other inserts.

The modular insert 145 is removably connected within the cage opening 85, 85a, 85b, the cage openings 85, 85a, 85b is sized and configured to receive the insert 145. The insert 145 matches or substantially matches the contours and/or the shape of the cage openings 85, 85a, 85b. The modular insert 145 comprises a superior surface and an inferior surface. The modular insert 145 comprises an insert opening 155, the insert opening 155 extends through the superior surface to the inferior surface of the insert 145.

The modular insert 145 comprises one or more keels 30, 35, 115, 130. The one or more keels 30, 35, 115, 130 may be disposed onto the superior surface 75 and/or inferior surface 80, extending between the anterior 63 and posterior surfaces 68. The one or more keels 30, 35, 115, 130 may be spaced apart from each other. The one or more keels 30, 35, 115, 130 may be aligned and/or offset from the adjacent keel 30, 35, 115, 130. The one or more keels 30, 35, 115, 130 may comprise one or more teeth 33, the one or more teeth 33 may be solid and/or each of the one or more teeth 33 may be solid. Alternatively, the one or more teeth 33 and/or each of the one or more teeth 33 may comprise capillary through holes 50. The anterior surface of the cage comprises a first opening and a second opening. In one embodiment, the modular insert 145 may comprise a dual keel, triple keel and/or a quad keel insert. The insert opening 155 may be disposed between the one or more keels 30, 35, 115, 130.

At least one of the superior surface 75, an inferior surface 80, an anterior surface 63, a posterior surface 68, medial surface 73 and/or a lateral surface 78 of the cage 25 may further comprise surface texturing 90, and/or any combination thereof as shown in FIGS. 4B, 8B-8C, 11, and 12A-12C. Accordingly, the cage 25 may further comprise a plurality of serrations 100, 105 as shown in FIG. 5. The plurality of serrations 100, 105 may be disposed adjacent to the first keel 30 and the second keel 35. The plurality of serrations 100, 105 may be disposed on the medial and/or lateral sides of the first keel 30 and the second keel 35. The plurality of serrations 100, 105 may be disposed on the superior 75 and/or inferior surfaces 80 extending upwardly.

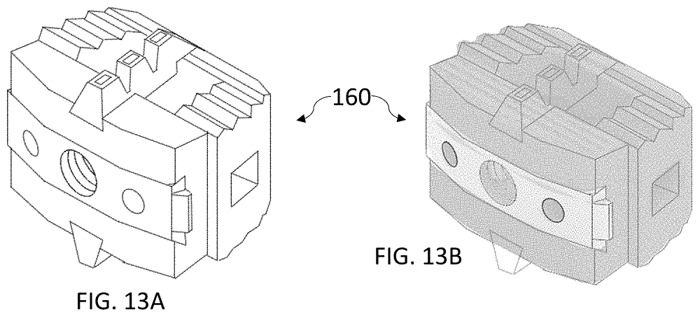

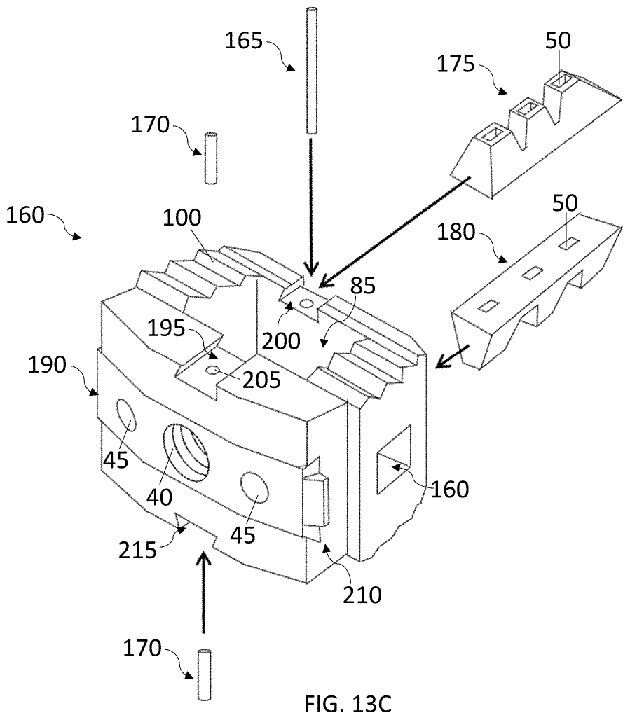

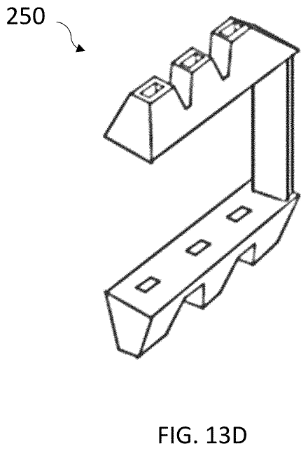

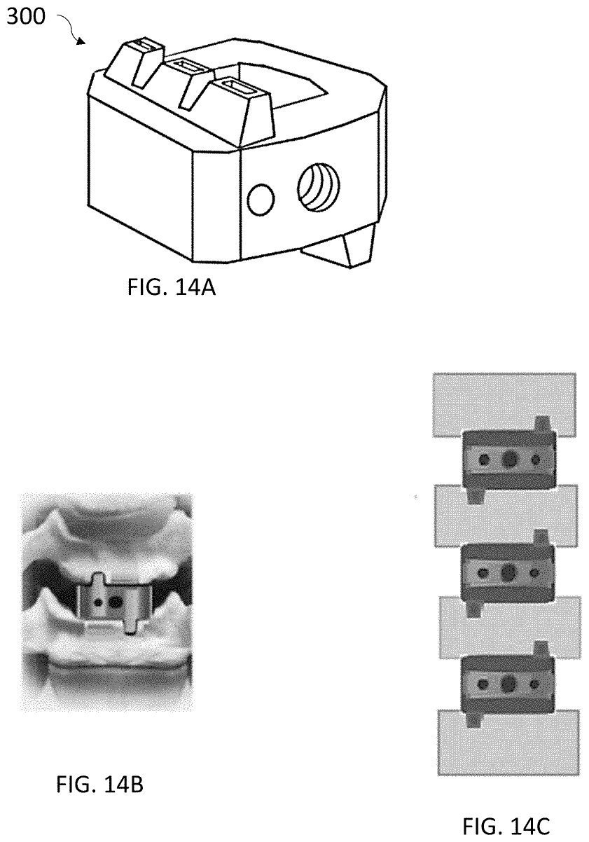

FIGS. 13A-13B depict an alternate embodiment of a modular keel ALIF cage system 160. The modular keel ALIF cage system 160 comprises: a cage 25, and one or more keels 175, 180. The modular keel ALIF cage system 160 may further comprise one or more of the following: frontal plate 190, dowel pins 165, 170, and/or a fixation screw (not shown). The cage 25 including a longitudinal axis 15, a superior surface 75, an inferior surface 80, an anterior surface 63, a posterior surface 68, medial surface 73 and/or a lateral surface 78. The cage 25 may further comprise a cage opening 85 extending from the superior surface 75 through the inferior surface 80 as shown in FIG. 4A-4B, FIG. 8A, FIGS. 12A-12C and 13A-13C. The cage opening 85 may extend through the superior surface 75 to the inferior surface 80. The cage opening 85 may be uniform and/or non-uniform. The cage opening 85 may further comprise a first cage opening 85a and a second cage opening 85b as shown in FIG. 8A. The first cage opening 85a comprising a different width from the second cage opening 85b. Each of the cage openings 85, 85a, 85b comprising a shape, each of the cage openings shapes may be the same or they may be different. Accordingly, the cage 25 may further comprise a plurality of serrations 100, 105 as shown in FIGS. 5 and 13A-13C. The plurality of serrations 100, 105 may be disposed adjacent to the one or more keels 175, 180. The plurality of serrations 100, 105 may be disposed on the medial and/or lateral sides of the one or more keels 175,180. The plurality of serrations 100, 105 may be disposed on the superior 75 and/or inferior surfaces 80 extending upwardly.

The cage 25 further comprising a one or more keel channels 195, 200, each of the one or more keel channels 195, 200 comprises a pin opening 200, the pin opening 200 is sized and configured to receive dowel pins 165, 170. The one or more keel channels 195,200 comprising a shape, the shape matching or substantially matching the shape of the one or more keels 175,180. The one or more keel channels 195, 200 may be disposed onto the superior surface 75 and/or the inferior surface 80 of the cage 25. The one or more keel channels 195,200 positioned adjacent to the cage opening 85, 85a, 85b. The cage 25 may further comprise a side opening 160, the side opening 160 extending from the medial 73 to lateral surface 78 of the cage 25. The side opening(s) 160 may engage with tools or other inserts. The cage 25 may further comprise plate channel 210, the plate channel 210 disposed on the anterior surface 63 of the cage 25. The plate channel 210 extending between the medial surface 73 and the lateral surface 78. The cage 25 may further comprise a plurality of openings 45, 50. At least one of the plurality of openings 45, 50 may comprise threads, at least one of the plurality of openings 45, 50 sized and configured for a fixation screw. At least one of the plurality openings 45, 50 may be sized and configured for a dowel pin.

The one or more keels 175,180 are removably connected within the one or more keel channels 195, 200, the one or more keel channels 195, 200 are sized and configured to receive the one or more keels 175,180. The one or more keel channels 195, 200 matches or substantially matches the contours of the one or more keels 175, 180. The one or more keels 175, 180 extending between the anterior 63 and posterior 68 surfaces. The one or more keels 175, 180 may be spaced apart from each other. The one or more keels 175, 180 may be aligned and/or offset from the adjacent keel 175, 180. The one or more keels 175, 180 may comprise one or more teeth 33, the one or more teeth 33 may be solid and/or each of the one or more teeth 33 may be solid. Alternatively, the one or more teeth 33 and/or each of the one or more teeth 33 may comprise capillary through holes 50. The anterior surface of the one or more keels 175, 180 may be flush with the anterior surface 63 of the cage 25. Alternatively, the anterior surface of the one or more keels 175, 180 may be offset with the anterior surface 63 of the cage 25. FIG. 13D depicts another alternative embodiment of a keel assembly 250 which can engage with and/or clamp around a cage in a manner to those similarly described herein.

The frontal plate 190 is desirably disposed into the plate channel 210 of the cage and may be removably connected. In various embodiments, the frontal plate may comprise a metal or plastic impaction plate that can be utilized in conjunction with a ceramic cage body (i.e., a cage body comprising silicon nitride) to provide for absorption and/or distribution of impacting forces for inserting the cage into and intervertebral space—especially where the cage body may be particularly susceptible to damage and/or fracture if directly impacted. The frontal plate 190 can further comprise a plurality of plate openings that are spaced apart, and concentrically aligned with the plurality of openings 45,50 of the cage 25. The frontal plate 190 comprises a body and a plurality of flanges. Each of the plurality of flanges are disposed onto medial and lateral surfaces of the frontal plate 190. The plurality of flanges extend outwardly from the medial and lateral surfaces of the frontal plate 190 and is positioned on the outside of the plate channel 210. The anterior surface of the frontal plate 190 may be flush with the anterior surface 63 of the cage 25. Alternatively, the anterior surface of the frontal plate 190 may be offset with the anterior surface 63 of the cage 25.

In the disclosed embodiments, various locking/securing mechanisms/means, if desired, are contemplated to help retain the device and/or components thereof in a specific adjustment (e.g., at least some distance of the respective engagement areas are adjusted). If desired, the implant device may have more or fewer components, and/or the number and distribution of links or other connecting features such as pins may vary accordingly. The devices may include exterior ridges, grooves, teeth, surface roughening, porous coatings or other treatments which enhance fixation to bone and/or bone ingrowth or on growth. The disclosed devices may further include one or more clamps, clips, clasps, braces, snapping mechanisms or other locking devices to hold the device in the compact configuration or in the expanded configuration. Such locking devices may be integral to the interbody device or may be entities separate from the interbody device. The various devices described herein may further include one or more biasing elements to bias the device toward the compact configuration or toward the expanded configuration.

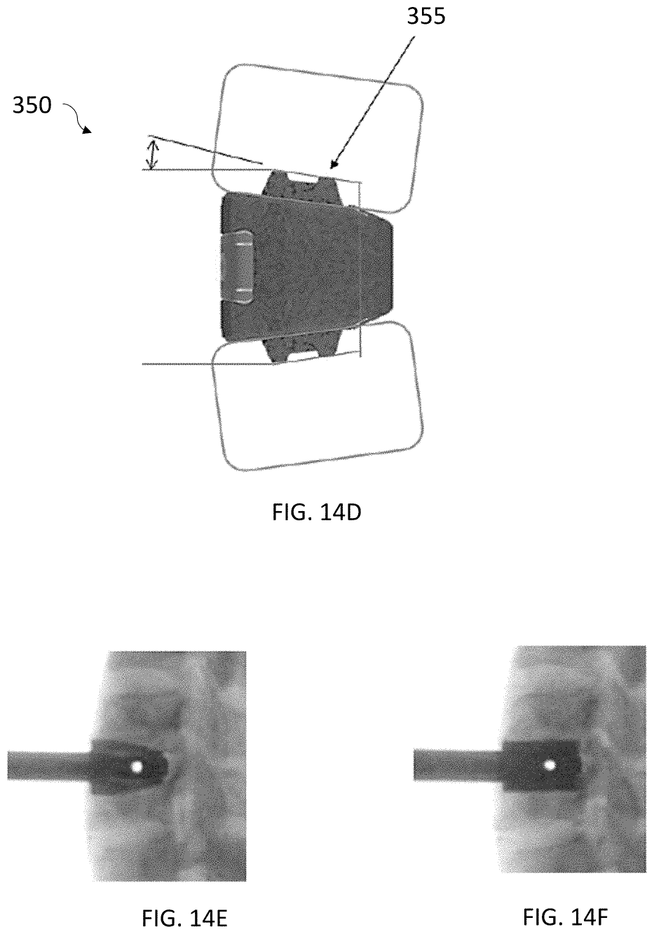

FIGS. 14A through 14C depict embodiments of a modular keel cage system 300 having a plurality of modular keel elements, wherein the keel elements are offset from each other. In this embodiment, one of both of the keel elements may optionally be arranged with an upper surface 355 positioned parallel to the endplates. FIG. 14B depicts the modular keel cage 300 in a single level spinal construct, while FIG. 14C depicts a plurality of modular keel cages in a multi-level spinal construct.

FIG. 14D depicts a modular keel cage system 350 including asymmetric (i.e., non-parallel) end plate surfaces, which in this embodiment can include angled end plates that provide for lordotic adjustment of the treated spinal level. FIG. 14E depicts a side view of the modular keel cage system 350 when implanted in a spinal level to alter the lordotic curvature of the patient's spine, while FIG. 14F depicts a side view of a modular keel cage having parallel endplates implanted in a spinal level for comparison.

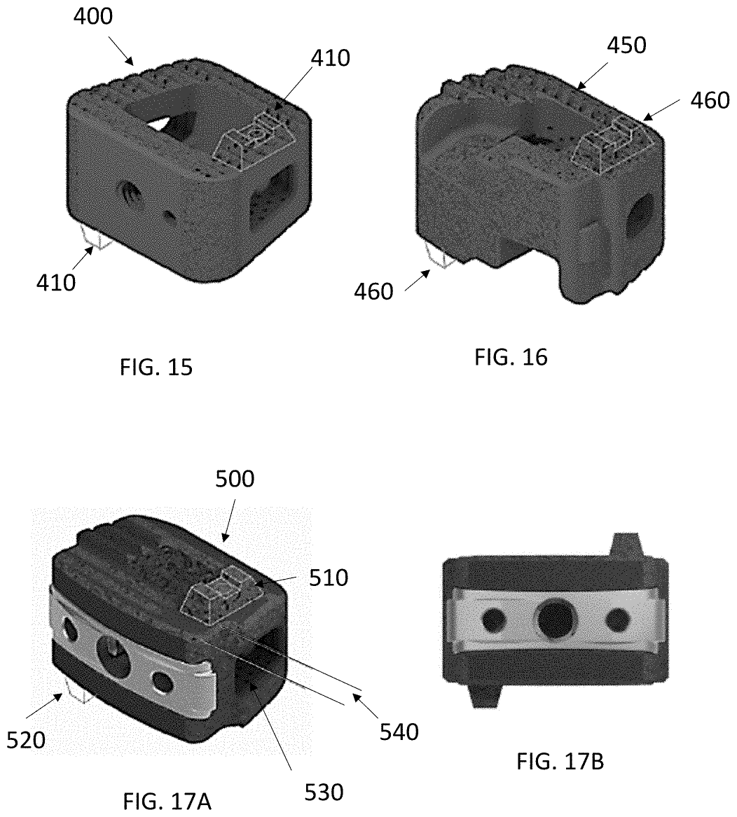

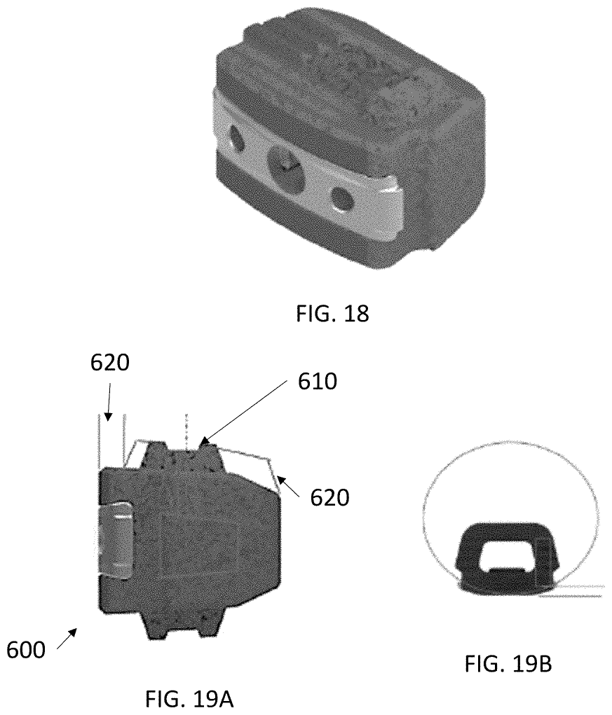

FIGS. 15 and 16 depict alternative embodiments of a modular cage system 400 and 450, each of these cages having offset modular keel elements 410 and 460. FIGS. 17A and 17B depict perspective and front plan views of another alternative embodiment of a modular keel cage 500 with offset keel elements 510 and an anterior impaction plate 520, as well as a lateral opening 530 through which bone graft and/or bony ingrowth may be visualized during the healing process. In this embodiment, the modular keel may be offset or otherwise spaced back or away from the anterior face of the cage by varying degrees (such spacing indicated by 540), such as by 1, 2, 3, 4 or 5 mm or portions thereof. FIG. 18 depicts a similar modular cage embodiment, without a lateral opening window.

FIGS. 19A and 19B depict side and top plan views of another exemplary embodiment of a modular cage system 600 wherein the modular keel elements 610 may include components of varying lengths, allowing for elongation of the keel assembly (up to the outline indicated as 620) if desired. Also indicated is an exemplary offset 640 for the keel, depending upon the chosen size and/or shape of modular component.

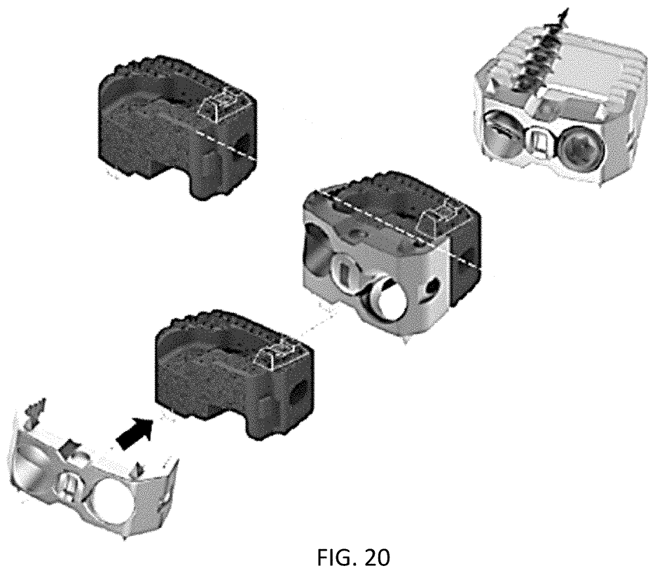

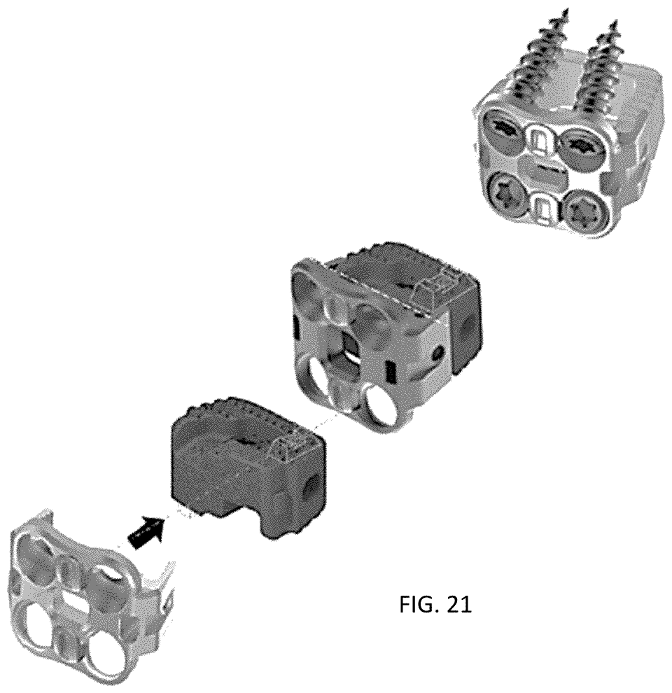

FIG. 20 depicts exploded and assembled views of another exemplary cage assembly with modular elements, wherein the cage includes 2 fixation screws. FIG. 21 depicts exploded and assembled views of another exemplary embodiment of a cage assembly with modular elements, wherein the cage includes 4 fixation screws.

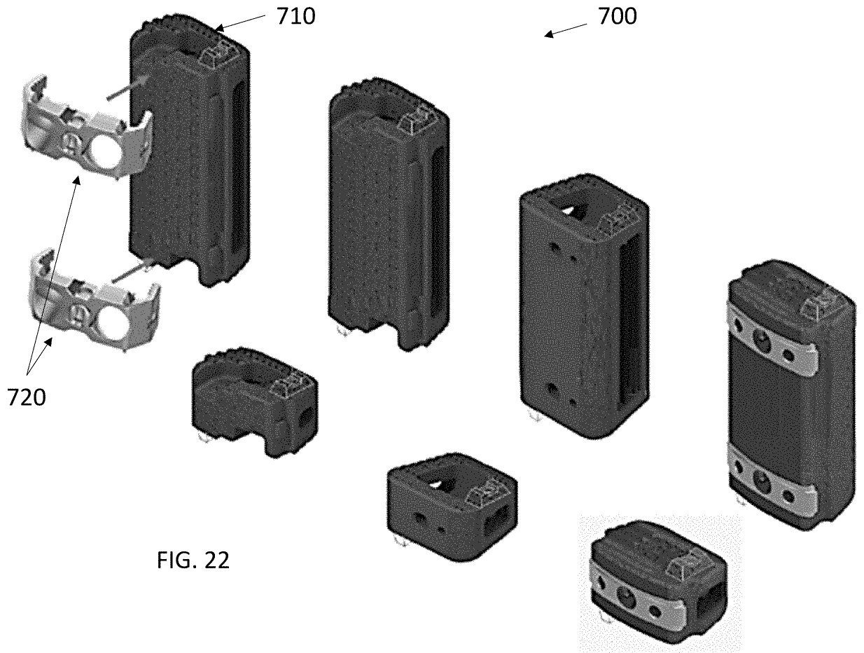

FIG. 22 illustrates perspective views of other exemplary embodiments of cage structures constructed according to the principles of the disclosure, where the cage structures may be particularly well suited for use as vertebral body replacements and/or used in corpectomy applications. The cage structures can include a cage shell 710 and impaction and/or fixation plates 720. When assembled, the cage structures may have a height that may range from, for example, about 4 mm to about 200 mm. Other heights are contemplated herein, including less than 4 mm or greater than 200 mm. The cage structures may include one or more holes (or openings), and/or fastening holes that may be configured to receive one or more bone fasteners (e.g., bone screws) to secure the cage structure to adjacent vertebrae. In this regard, the fastening holes may optionally be angled so as to guide the bone fasteners toward and into the vertebrae. The cage structure may further include one or more openings to accommodate graft materials, which may include an opening formed at, for example, a center portion of the cage structure. The openings may be laterally surrounded and defined by inner wall surfaces. Any of the upper and/or lower or walls surfaces may have a surface pattern, which may be configured to directly contact a surface of the adjacent vertebra during implantation, with such surface pattern potentially establishing and/or promoting bone growth and/or resisting movement (e.g., departure, slippage, or the like), as described above.

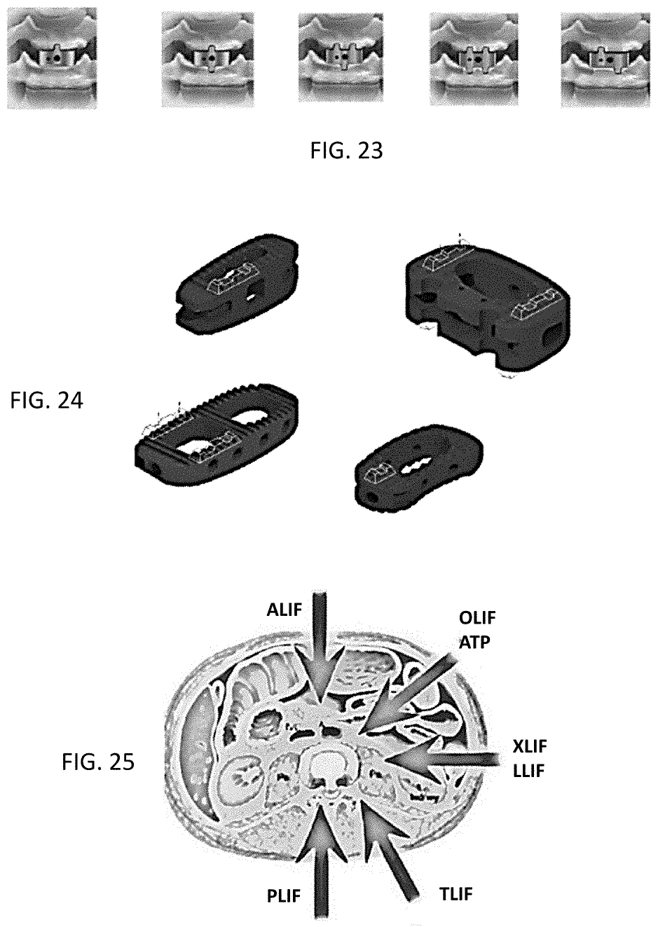

FIG. 23 depicts front plan views of various keel positions that can be accommodated by a single modular keel system by simple addition/substitution of muscular keel elements. FIG. 24 depicts various embodiments of interbody spacers that can accommodate and/or incorporate modular keel elements.

The various components described herein may be constructed of a variety of materials and/or material combinations, including the use of known medical grade materials for implants, including but not limited to, metal (e.g., titanium), metal alloy (e.g., titanium alloy), plastic, ceramic, elastomers, carbon fiber reinforced polymers, polyetheretherketone (PEEK), tricalcium phosphate, hyroxyaptaite, silicon nitride, autograft, allograft, and/or any combinations thereof.

In various embodiments, the disclosed system may include various modular cage features, including one or more cage bodies and one or more modular keel features. For example, proper configuration of the modular cage system components can be used to minimize inventory of parts. If desired, the modular cage system may provide an adjustable footprint, including where a closed loop geometry may be implemented, an open loop geometry may be implemented, and/or a hybrid closed-open loop geometry may be implemented.

In various embodiments, a modular cage system may comprise one or a plurality of cage bodies and one or more modular keel features. Each of the cage bodies may have substantially the same shape and varying (e.g., increasing or decreasing) (e.g. height, width, length, surface angle (e.g., angle of superior surface along posterior-anterior and/or lateral directions of cage body, and/or angle of inferior surface along posterior-anterior and/or lateral directions of cage body)), so that a plurality of cage bodies may be nested together to form a unitary configuration of the modular cage system, such as by nesting one inside another. One or more of the cage bodies may have a different shape and/or size than the other cage bodies. The cage bodies and modular keel element(s) may be selected and nested together to form a cage system that matches the size, shape, contours, etc. of the adjacent vertebrae surfaces. Each of the cage bodies and/or modular keel element(s) may be made of a single material or combination of various materials for, for example, radio-opaque and/or strength effects. The cage bodies and/or modular keel element(s) may be made of the same or different materials. The modular cage system may include, for example, one, two, three, four, or more modular keel element(s) which attach to one or more cage bodies. Desirably, the cage body may be nested with the one or more modular keel element(s) to together form a unitary configuration of the modular cage system.

The cage bodies may have a graft chamber, whose dimensions and position may be varied by varying the thicknesses and/or shapes of the walls of the respective cage body. For instance, by making one of the four walls of the cage body much thicker than the other three walls, the center of the graft chamber may be shifted away from the thicker wall. Further, by altering the inner contours of the walls of a cage body, the shape of the graft chamber may be selectively determined. The outer contours of the walls of one or more of the cage bodies may be varied to form cage bodies based on the particular anatomy of a patient.

If desired, the components of the modular cage system may optionally include one or more end caps (not shown), which may comprise an insert portion and/or a rim portion, such as disclosed in co-pending U.S. patent application Ser. No. 17/341,297, the disclosure of which is incorporated herein by reference. The thickness, size and/or shape of the wall portions that form the insert portion may be predetermined so as to selectively determine the position, shape, and/or size of the graft chamber in the cage body. For instance, the walls of the insert portion may be varied in terms of size and shape, including, for example, height, width, length, surface angles, so as to determine the shape, position and size of the graft chamber in the cage body when the end cap is attached to the cage body. Similarly, the thickness, size and/or shape of the rim portion may be varied to, for example, match anatomical requirements for particular applications of the cage system. For instance the height of the walls that form the rim portion may be decreased (or increased) in the posterior (or anterior) direction, so as to provide better fit in vertebral interbody applications. The rim portion may be configured to contact and engage a vertebral body. In this regard, the surface of the rim portion may be contoured to match the shape of the vertebral body. The surface may include keel elements or similar bone interface members that may be configured to aggressively grip against the bony surface of the adjacent vertebral body.

In one exemplary surgical procedure utilizing the disclosed devices, a surgeon can first surgically access a targeted region of a patient's spine and remove some or all of one or more interspinal discs and/or other spinal structures (as is well known in the art). The surgeon can then utilize one or more surgical trial devices to determine the size and/or shape of the vacated disc space, as well as determine the laxity of the annulus and/or surrounding soft tissues, allowing the surgeon to determine a desired size and/or shape of fusion implant or similar device to place within the disc space. The surgeon may utilize a keel or breaching chisel or similar device to create a channel into the upper and/or lower vertebral body surfaces. In some instances, a surgeon may optionally choose to use a keel path pilot hole drill guide and/or temporary fixation pins. Once the vertebral surfaces have been prepared, an appropriate implant may be inserted, which may include the introduction of bone graft material and/or other substances (as well as the placement of supplemental fixation hardware, if desired), and the surgical incision may be closed in a known manner.

In various embodiments, the ability to modify the various keel elements of a given cage design may be particularly useful to a surgeon addressing highly degenerative and/or atypical spinal anatomy. In such a case, an appropriate keel design may be selected to adequately secure the implant in a desired position while accommodating anatomical features such as weakened and/or missing bony support regions in some of all of the targeted vertebral bodies. Desirably, the present embodiment will allow a surgeon to particularize an implant at the time the surgeon can directly assess the surgical field, including allowing for direct knowledge and open visualization of the patient anatomy at the time the keel elements and/or other implant components are being selected and/or assembled.

While many of the disclosed embodiments are described in connection with an ALIF cage system and related surgical procedure, it should be understood that various other implant systems and/or systems suitable for alternative surgical approaches may benefit from various teachings of the present invention (and/or portions thereof), including systems and/or devices used in conjunction with various discectomy and fusion procedures such as, for example, anterior cervical interbody fusion (ACIF), anterior lumbar interbody fusion (ALIF), direct lateral interbody fusion (DLIF) (also known as XLIF), posterior lumbar interbody fusion (PLIF), and transforaminal lumbar interbody fusion (TLIF), as well as the surgical approaches depicted in FIG. 25 and/or many other techniques known by those of ordinary skill in the art.

In various embodiment, method(s) for manufacturing the disclosed devices and/or implanting the device into a spine are contemplated and are part of the scope of the present application.

Devices and/or components thereof that are in communication with each other need not be in continuous communication with each other, unless expressly specified otherwise. In addition, devices that are in communication with each other may communicate directly or indirectly through one or more intermediaries.

Although process steps, method steps, or the like, may be described in a sequential order, such processes and methods may be configured to work in alternate orders. In other words, any sequence or order of steps that may be described does not necessarily indicate a requirement that the steps be performed in that order. The steps of the processes or methods described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices which are not explicitly described as having such functionality or features.

While the disclosure has been described in terms of exemplary embodiments, those skilled in the art will recognize that the disclosure can be practiced with modifications in the spirit and scope of the appended claims. These examples are merely illustrative and are not meant to be an exhaustive list of all possible designs, embodiments, applications or modifications of the disclosure. While embodiments and applications of the present subject matter have been shown and described, it should be apparent to those skilled in the art that many more modifications are possible without departing from the inventive concepts herein. The subject matter, therefore, is not to be restricted except in the spirit of the appended claims. Thus, while embodiments and applications of the present subject matter have been shown and described, it should be apparent that other embodiments, applications and aspects are possible and are thus contemplated and are within the scope of this application.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

I/We claim:

1. An intervertebral cage system comprising:
a cage, the cage including a longitudinal axis, a superior surface, an inferior surface, an anterior surface, a posterior surface, a medial surface, and a lateral surface, the anterior surface comprising a first opening and a second opening; the first opening being larger than the second opening; the first opening comprising threads to engage a fixation screw; and
a first keel and a second keel, the first keel disposed onto a superior surface, the second keel disposed onto an inferior surface, the first keel and the second keel extend between the anterior and posterior surfaces of the cage, each of the first and second keels comprise a plurality a teeth.

2. The intervertebral cage system of claim 1, wherein the ALIF cage system further comprises a third keel, the third keel is disposed onto the superior surface, the third keel is spaced apart from the first keel.

3. The intervertebral cage system of claim 3, wherein the ALIF cage system further comprises a fourth keel, the fourth keel is spaced apart from the second keel.

4. The intervertebral cage system of claim 1, wherein the plurality of teeth of each of the first keel and second keels comprises capillary through holes.

5. The intervertebral cage system of claim 1, wherein the cage comprises a first side channel and a second side channel, the first side channel disposed onto the medial surface of the cage, the second side channel disposed on the lateral surface of the cage, the first and second side channels extending between the superior and inferior surfaces of the cage.

6. The intervertebral cage system of claim 1, wherein the cage further comprises a cage opening, the cage opening extending through the superior to the inferior surface of the cage.

7. The intervertebral cage system of claim 1, wherein at least one of the superior surface, an inferior surface, an anterior surface, a posterior surface, a medial surface, a lateral surface, the anterior surface of the cage further comprises surface texturing.

8. A modular keel intervertebral cage system comprising:
a cage, the cage including a longitudinal axis, a superior surface, an inferior surface, an anterior surface, a posterior surface, a medial surface, and a lateral surface, the anterior surface comprising a first opening and a second opening; the first opening being larger than the second opening; the first opening comprising threads to engage a fixation screw, the cage further comprising a cage opening, the cage opening extending through the superior to inferior surfaces; and
an insert, the insert disposed into the cage opening, the insert comprising a first keel and a second keel, the first keel disposed onto a superior surface, the second keel disposed onto an inferior surface, the first keel and the second keel extend between the anterior and posterior surfaces of the cage, each of the first and second keels comprise a plurality a teeth.

9. The modular keel intervertebral cage system of claim 8, wherein the ALIF cage system further comprises a third keel, the third keel is disposed onto the superior surface, the third keel is spaced apart from the first keel.

10. The modular keel intervertebral cage system of claim 9, wherein the ALIF cage system further comprises a fourth keel, the fourth keel is spaced apart from the second keel.

11. The modular keel intervertebral cage system of claim 8, wherein the plurality of teeth of each of the first keel and second keels comprises capillary through holes.

12. The modular keel intervertebral cage system of claim 8, wherein the cage comprises a first side opening and a second side opening, the first side opening disposed onto the medial surface of the cage, the second side opening disposed on the lateral surface of the cage.

13. A modular keel intervertebral cage system comprising:
a cage, the cage including a longitudinal axis, a superior surface, an inferior surface, an anterior surface, a posterior surface, a medial surface, and a lateral surface, the anterior surface comprising a first opening and a second opening; the first opening being larger than the second opening; the first opening comprising threads to engage a fixation screw, the cage further comprising a cage opening and one or more keel channels, the cage opening extending through the superior to inferior surfaces of the cage, the one or more keel channels disposed onto a portion of the superior and inferior surfaces of the cage,
a first keel and a second keels, the first and second keels disposed within the one or more keel channels of the cage, the first keel and the second keels extend between the anterior and posterior surfaces of the cage, each of the first and second keels comprise a plurality a teeth.

14. The modular keel intervertebral cage system of claim 13, wherein the ALIF cage system further comprises a third keel, the third keel is disposed onto the superior surface, the third keel is spaced apart from the first keel.

15. The modular keel intervertebral cage system of claim 14, wherein the ALIF cage system further comprises a fourth keel, the fourth keel is spaced apart from the second keel.

16. The modular keel intervertebral cage system of claim 13, wherein the plurality of teeth of each of the first keel and second keels comprises capillary through holes.

17. The modular keel intervertebral cage system of claim 13, wherein the cage comprises a first side opening and a second side opening, the first side opening disposed onto the medial surface of the cage, the second side opening disposed on the lateral surface of the cage.

18. The modular keel intervertebral cage system of claim 13, wherein at least one of the superior surface, an inferior surface, an anterior surface, a posterior surface, a medial surface, a lateral surface, the anterior surface of the cage further comprises surface texturing.

19. The modular keel intervertebral cage system of claim 13, wherein the anterior surface of the cage further comprises a plate channel.

20. The modular keel intervertebral cage system of claim 19, wherein modular keel ALIF cage system further comprises a frontal plate, the frontal plate disposed within the plate channel of the cage.
